(19) United States
(12) Patent Application Publication
(10) Pub. No.: US 2017/0119263 A1
(43) Pub. Date: May 4, 2017

(54) System and Method for Deriving a Pulse Wave Velocity-Blood pressure Transform

(71) Applicant: Sharp Laboratories of America (SLA), Inc., Camas, WA (US)

(72) Inventor: Fredrick Hill, Portland, OR (US)

(21) Appl. No.: 14/932,019

(22) Filed: Nov. 4, 2015

(51) Int. Cl. A61B 5/021; A61B 5/022; A61B 5/00

(52) U.S. Cl. CPC A61B 5/02125; A61B 5/7221; A61B 5/7253; A61B 5/02233; A61B 5/7246

(57) ABSTRACT

A system and method is provided for deriving a pulse wave velocity-blood pressure (PWV-BP) transform. The method provides a general population PWV-BP transform comprising an average, population correlation of PWV measurements to BP measurements, where the selected average population shares common characteristics with a selected individual. The personal PWV-BP transform is based upon PWV and BP measurements for the first individual. If the personal PWV-BP transform is determined to be invalid, the general population PWV-BP is used to correlate the current PWV measurement for the first individual, to an estimated BP value for the first individual. The method modifies the general population PWV-BP transform using the at least a first PWV and BP measurement, and updates the general population PWV-BP transform. If a Sufficient number of simultaneous PWV and BP measurements are taken, and determined to be statistically valid, a personal PWV-BP transform can be established.

### BACKGROUND OF THE INVENTION

1. Field of the Invention

This invention generally relates to blood pressure measurement and, more particularly, to a system and method for deriving blood pressure from pulse wave velocity measurements.

2. Description of the Related Art

Blood pressure is a key vital sign used in the diagnosis and monitoring of a host of diseases [1]. The gold standard in blood pressure monitoring is arterial catheterization, in which a catheter is inserted into an artery and a continuous pressure waveform is produced. That waveform represents pressure at the catheter tip relative to atmospheric and bears a large amount of information regarding the state of the arterial system and patient health in general. The arterial pressure waveform supports a host of metrics, including the well-known systolic and diastolic pressures, but is handicapped for general use by its invasive nature.

In the mid nineteenth century. Marey pioneered a mechanical device which non-invasively charts blood pressure (typically from brachial or radial arteries). Shortly after, a method came into practice in which absolute blood pressure is estimated by observing pressure oscillations on an artery compressed by an inflatable cuff while slowly releasing the cuff pressure. This method has become known as the “oscellometric” method and is the basis for nearly oil automated blood pressure measurements in use today. While the oscellometric method is remarkable for its early discovery and widespread application, the measurement has high uncertainly and error and requires about one half minute to produce a single measurement of diastolic and systolic blood pressure. Furthermore, the required arterial compression necessitates a recovery period and the inherent pump size and noise make this method awkward for worn devices.

In the early twentieth century Korotkoff developed an auscultatory method for estimating blood pressure, using proximal compression of the artery. This method was widely adopted by practitioners and is still considered the most reliable method of noninvasively estimating systolic and diastolic blood pressure. It is the reference measurement for the ANSI/ISO/AAMI SP-10 standard. While this measurement has been shown to be more accurate than the oscillometric method, it has not achieved widespread adoption in automated form and requires a skilled human operator. Like the oscillometric method—it requires a long measurement interval and recovery period.

In recent years, consensus has developed that a strong correlation exists between arterial pulse wave velocity (PWV) and systolic and diastolic blood pressure [2]. A PWV measurement involves a combination of simultaneous electrocardiography (ECG or EKG) and photoplethysmography (PPG) measurements. Electrocardiography is the process of recording the electrical activity of the heart over a period of time using electrodes placed on a patient's body. These electrodes detect the tiny electrical changes on the skin that arise from the heart muscle depolarizing during each heartbeat. During each heartbeat, a healthy heart has an orderly progression of depolarization that starts with pacemaker cells in the sinoatrial node, spreads out through the atrium, passes through the atrioventricular node down into the bundle of His and into the Purkinje fibers spreading down and to the left throughout the ventricles. This orderly pattern of depolarization gives rise to the characteristic ECG tracing.

Photoplethysmorgraphy is a method of measuring the perfusion of blood to the dermis and subcutaneous tissue by illuminating the tissue at the surface and observing variations of the light. With each cardiac cycle the heart pumps blood to the periphery. The change in blood volume caused by the pressure pulse of the cardiac cycle is detected by illuminating the skin with a light-emitting diode (LED) and measuring the amount of light cither transmitted or reflected to a photodiode. The resulting waveform characterizes the relative blood volume of the tissue over time.

Attempts to transform PWV measurements to blood pressure with reasonable (i.e., AAMI/ANSI/ISO SP10) accuracy have been successful and at least one product is now available and approved for hospital use [3, 4]. PWV-based blood pressure (PWV-BP) addresses many limitations of the oscillometric and auscultatory methods. It requires no arterial compression, no cuff, and no recovery interval A measurement can be formed on every arterial pulse and integrated over time to reduce measurement uncertainty. In some modalities, it is possible to collect the measurement in a worn device and continuously update the blood pressure estimate. However, PWV-based blood pressure requires a transform from PWV to Mood pressure and that transform varies from patient to patient, over populations, and over time [6]. Some PWB-BP designs require frequent calibration—at least once per 8 hours [3]. Others eschew calibration entirely and base the transform on population norms [5].

To yield an effective measurement modality, the transform from PWV to BP must be considered in the context of the use case of the measurement device. The transform can be crafted in terms of population norms, but because patients may not reflect the norms of their populations, a calibrated transform would yield better measurement accuracy. In some use cases, calibration is simply not feasible. In others, calibration may be sporadic. A key challenge to the PWV-BP measurement is producing an accurate transform from PWV to BP. Two approaches are now prominent: calibrations collected over short intervals and repeated often (e.g., every eight hours) and use of transforms based only on population norms. Neither is ideal for the home use case. It would be advantageous if a uniform objective framework existed in which PWV-to-BP transform estimates could transition from a basis in general populations to a personal transform and back to general population basis according to the calibration data available and the PWV measurement being transformed.

1. Nichols, W., el al, McDonalds Blood Flow in Arteries, 6th Edition. 2011, CBC Press.

2. Gesche, H., D. Grosskurth. G, Kuchler, A. Patzak, “Continuous blood pressure measurement by using the pulse transit time: comparison to a cuff-based method”, European Journal of Applied Physiology. DOI 10.1007/s00421-011-1983-3, May 2011.

3. Banet, M., et at “Body-worn System for measuring continuous non-invasive blood pressure (cNIBP)”, US Patent US20100160795 A1, Pub. Date Jun. 24, 2010.

4. Sotera Wireless, Inc., Visi Mobile Monitoring System. 510K Summary, Prepared Jun. 11, 2013.

5. Scanadu Press Release, “Scanadu Seizures $35 Million in Series B Funding to Advance Go-to-Market Plans”, Apr. 27, 2015, https://www.scanadu.com/pr/.

6. Zhang, G., et al, “Assessing the Challenges of a Pulse Wave Velocity Based Blood Pressure Measurement in Surgical Patients”, IEEE EMBC, 2014.

7. Poon, C., et al, “Cuff-less and Noninvasive Measurements of Arterial Blood Pressure by Pulse Transit Time”, IEEE EMBS, 2005.

8. Hermelling, E., “Local Pulse Wave Velocity Determination: The Arterial Distension Waveform from Foot to Crest”, PhD Thesis, 2009.

9. Boutouyrie, P., Vermeersch, S. J., “Determinants of pulse wave velocity in healthy people and in the presence of cardiovascular risk factors: establishing normal and reference values”, European Heart Journal (2010) 31, 2338-2350.

10. Devore, J., Probability and Statistics for Engineering and the Sciences, Brooks/Cole, 2012.

### SUMMARY OF THE INVENTION

A blood pressure measurement based on Pulse Wave Velocity (PWV) has many appealing qualities. The measurement requires no arterial compression and no recovery period. The measuring device can be small and inconspicuous and might even be worn for long periods. The measurement is very fast, producing a new blood pressure estimate on every heartbeat. As such, it is possible to estimate measurement uncertainty and reduce measurement error by methods like time averaging, uncertainty weighting, and median filters.

The method described here evolves the PWV-to-blood pressure (PWV-BP) transform through a strategy of successive refinement. This approach supports both a longer time interval for the collection of calibration data and a larger, more diverse collection of calibration data than might be found using short-term calibration. As such the calibration data, over lime, belter represent the mean state of the patient than data resulting from shorter-term calibration windows. Furthermore, personalized transforms offer improved accuracy over those derived purely from population indices. As such, the method offers the promise of improved accuracy, to the extent supported by the sample statistics. Finally, the focus on automatic and incremental refinement based on screened data and transforms is consistent with the home use case, where measurements may be taken sporadically, over long periods of time, and may occasionally be of poor quality.

Calibration measurements are taken, screened for uncertainty, and used to refine an initial population transform. As calibration points accumulate, a personalized trial transform is formed and evaluated on its sample statistics. Once that transform achieves viability, it is used as the basis of the PWV-BP transform.

Accordingly, a method is provided for deriving a PWV-BP transform. The method provides a general population PWV-BP transform comprising an average population correlation of PWV measurements to BP measurements, where the selected average population shares common characteristics with a selected first individual. The method determines the validity of a personal PWV-BP transform. The personal PWV-BP transform is based upon PWV and BP measurements for the first individual. If the personal PWV-BP transform is determined to be invalid, the general population PWV-BP is used to correlate the current PWV measurement for the first individual, to an estimated BP value for the first individual.

In one aspect, the method simultaneously takes at least a first PWV measurement and a first BP measurement for the first individual. Typically, several measurements are taken. The method modifies the general population PWV-BP transform using at least the first PWV measurement and the first BP measurement, and saves the modified general population PWV-BP transform as an updated general population PWV-BP transform. If a sufficient number of simultaneous PWV measurements and BP measurements are taken for the first individual a personal PWV-BP transform can be established, subject to statistical tests on the measurements.

If the personal PWV-BP transform is determined to be valid, it can be used to correlate the current PWV measurement for the first individual, to the BP estimate for the first individual. Otherwise, the personal PWV-BP transform may be determined to be invalid if the current PWV measurement fails to meet a statistical limit derived from the current PWV measurement for the first individual and the plurality of simultaneous PWV measurements and BP measurements for the first individual used to establish this personal PWV-BP transform. That is, the personal PWV-BP transform may be deemed invalid if the plurality of simultaneous PWV measurements and BP measurements for the first individual fail to meet a statistical limit.

Additional details of the above-described method and a device for correlating PWV and BP measurements are presented below.

### BRIEF DESCRIPTION OF THE DRAWINGS

FIG. 1 is a schematic block diagram of a device for correlating pulse wave velocity (PWV) and blood pressure (BP) measurements.

FIG. 2 is a graph of systolic blood pressure (SBP) vs. PWV measurements depicting a calibration set (dots), estimated PWV-to-BP transform, BP estimates (x), and BP estimate with a confidence interval.

FIG. 3 in a graph depicting a family of transform curves derived from age-decade populations in a large study (n=11,092) [9].

FIG. 4 illustrates waveforms of an exemplary PWV measurement that may be acquired on every arterial pulse.

FIG. 5 is a block diagram depicting an exemplary realization of a PWB-BP calibration measurement.

FIG. 6 illustrates the derivation and application of the PWV-BP transform.

FIG. 7 is a flowchart illustrating a method for deriving a PWV-BP transform.

### DETAILED DESCRIPTION

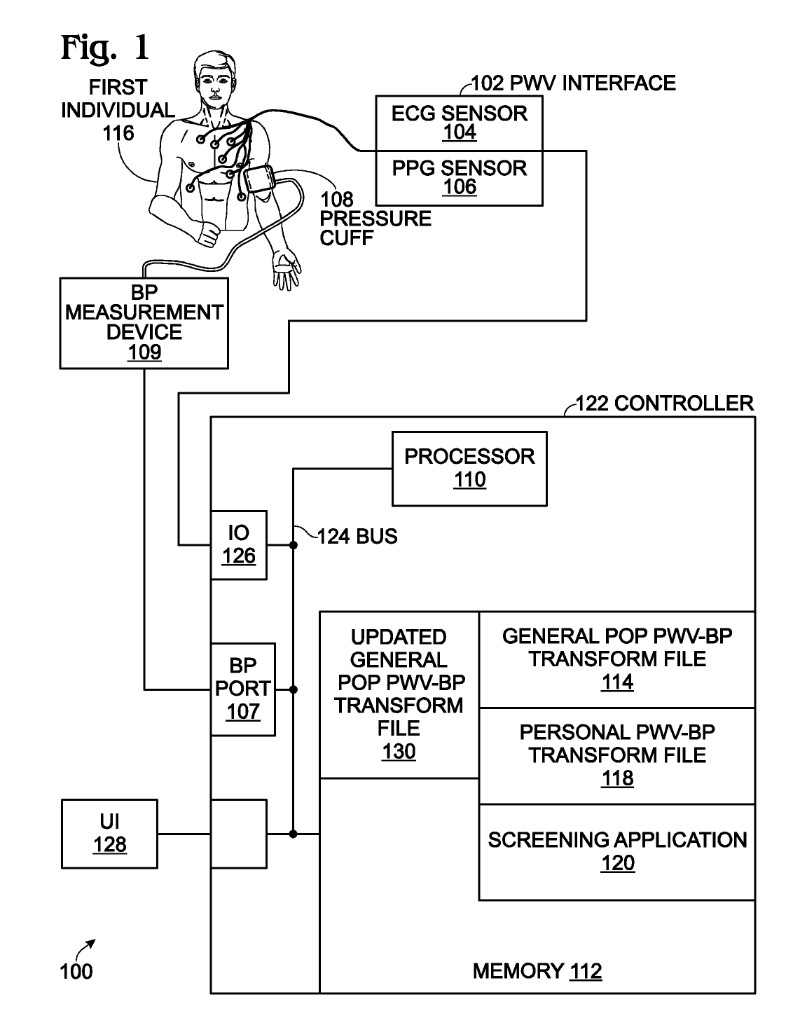

FIG. 1 is a schematic block diagram of a device for correlating pulse wave velocity (PWV) and blood pressure (BP) measurements. The device 100 comprises a PWV measurement interface 102 comprising an electrocardiogram (ECG) sensor 104 and a photoplethysmoeraphy (PPG) sensor 106 for measuring ECG and PPG signals. Typically, the PPG sensor 106 comprises a light emission device and a light sensing device (not shown) for detecting changes in optical transmittance of an illuminated test subject body. Typically, the ECG sensor 104 comprises at least two electrodes (not shown).

The device 100 also includes a BP port 107 to accept BP measurements. As shown, a BP measurement device 109 may be connected to the BP port 107 to supply BP measurements, however taken. In one aspect, the BP measurement device 109 collects BP measurements when connected to a pressure cuff 108. As device 100 is typically used, the collection of BP measurements is a relatively rare occurrence, and in some aspects the BP port 107 is optional. The device further comprises a processor 110. A non-transitory memory 112 includes a general population PWV-BP transform file 114 with average population correlations of PWV measurements to BP measurements. The selected average population shares common characteristics with the selected first individual (patient) 116. For example, the first individual 116 may be characterized as a 35 year Caucasian male, with a height of 5 feet eight inches, with a weight of 180 pounds. The memory 112 also comprises a personal PWV-BP transform file 118. The personal PWV-BP transform file 118 is based upon PWV and BP measurements for a first individual 116, In addition, the memory 112 comprises a screening application 120 enabled as a sequence of processor instructions for determining the validity of the personal PWV-BP transform file 118. The screening application 120 selects the general population PWV-BP transform file 114 when the personal PWV-BP transform file 118 is determined to be invalid, and uses the general population PWV-BP file to correlate a current PWV measurement for the first individual 116, to an estimated BP value for the first individual. As explained below, the general population PWV-BP transform may be updated to more closely match the first individual, creating a personalized form of the general population PWV-BP file.

Portions of device 100, including the processor 110, memory 112, screening application 120 may be referred to as a computing device or controller 122. The controller 122 may also include a bus 124, input/output (IO) port 126, and user interface (UI) 128. The communication bus 124 may, for example, be a Serial Peripheral Interface (SPI), an Inter-Integra ted Circuit (IZC), a Universal Asynchronous Receiver/Transmitter (UART), and/or any other suitable bus or network. Although the drawing implies that the components of the controller 122 are collocated in the same device, in some aspects various components may be located outside the device, communicating with other components via a wired or wireless connection.

The memory 122 may include a main memory; a random access memory (RAM), or other dynamic storage devices. These memories may also be referred to as a computer readable medium. Such a medium may take many forms, including but not limited to, non-volatile media, volatile media, and transmission media. Non-volatile media includes, for example, optical or magnetic disks. Volatile media includes dynamic memory. Common forms of computer readable media include, for example, a floppy disk, a flexible disk, hard disk, magnetic tape, or any other magnetic medium, a CD-ROM, any other optical medium, punch cards, paper tape, any other physical medium with patterns of holes, a RAM, a PROM, and EPSOM, a FLASH-EEPROM, any other memory chip or cartridge, or any other medium from which a computer can read. The execution of the sequences of instructions contained in a computer-readable medium (i.e. screening application 120) may cause the processor 110 to perform some of the steps of determining PWV*BP transform validity. Alternately, some of these functions may be performed in hardware (not shown). The practical implementation of such a computer system would be well known to one with skill in the art. In one aspect, the processor 110 is an ARM processor using a reduced instruction set computing (RISC) architecture.

The user interface 128 and 10 port 126 may incorporate a display, a modem, an Ethernet card, or any other appropriate data communications device such as USB. The physical communication links may be optical, wired, or wireless. The user interface 128 may incorporate a keypad or a cursor control device such as a mouse, touchpad. touchscreen, trackball, stylus, or cursor direction keys.

The controller 122 may be considered a type of special purpose computing system, and as such, can be programmed, configured, and/or otherwise designed to comply with one or more networking protocols. According to certain embodiments, the controller 122 may be designed to work with protocols of one or more layers of the Open Systems Interconnection (OSI) reference model, such as a physical layer protocol, a link layer protocol, a network layer protocol, a transport layer protocol, a session layer protocol, a presentation layer protocol, and/or an application layer protocol. For example, IO 126 may include a network device configured according to a Universal Serial Bus (USB) protocol, an Institute of Electrical and Electronics Engineers (IEEE) 1394 protocol, an Ethernet protocol, a T1 protocol, a Synchronous Optical Networking (SONET) protocol, a Synchronous Digital Hierarchy (SDH) protocol, an Integrated Services Digital Network (ISDN) protocol, an Asynchronous Transfer Mode (ATM) protocol, a Point-to-Point Protocol (PPP), a Point-to-Point Protocol over Ethernet (PPPoE), a Point-to-Point Protocol over ATM (PPPoA), a Bluetooth protocol, au IEEE 802.XX protocol, a frame relay protocol, a token ring protocol, a spanning tree protocol, and/or any other suitable protocol.

The controller 122 may provide a direct connection to a remote server via a direct link to a network, such as the Internet. Connection may be provided through, for example, a local area network (such as an Ethernet network), a personal area network, a wide area network, a private network (e.g., a virtual private network), a telephone or cable network, a cellular telephone connection, a satellite data connection, or any ether suitable connection.

In certain embodiments, a host adapter is configured to facilitate communication between controller 122 and one or more network or storage devices via an external bus or communications channel. Examples of host adapters include, without limitation, Small Computer System Interface (SCSI) host adapters. Universal Serial Bus (USB) host adapters, IEEE 1394 host adapters, Advanced Technology Attachment (ATA), Parallel ATA CP ATA), Serial ATA (SATA), and External SATA (eSATA) host adapters. Fibre Channel interface adapters, Ethernet adapters, or the like.

In one aspect, the screening application 120 accepts data for at least a first PWV measurement and a first BP measurement, simultaneously taken for the first individual 116, and creates an updated general population PWV-BP transform file 130 stored in memory 112. The updated general population PWV-BP transform file 130 includes the general population PWV-BP transform file 114 as modified by at least the first PWV measurement and the first BP measurement.

In one aspect, the screening application 120 accepts data for a plurality of simultaneous PWV measurements and BP measurements taken for the first individual, and creates the personal PWV-BP transform file 118 in response to the plurality of PWV and BP measurements. The personal PWV-BP transform file 128 includes a plurality of PWV-to-BP data points, cross-referenced to timestamps, and the screening application 120 creates the personal PWV-BP transform file in response to the PWV-to-BP data point timestamps and a PWV-to-BP data point count (a predetermined sufficient number of data points). In one aspect, the screening application 120 creates the personal PWV-BP transform file 118 by collecting a PWV measurement as a statistical mean over many arterial pulses within the time interval of the PWV measurement. In another aspect, the personal PWV-BP transform file 118 includes a PWV-to-BP sample set, with PWV-to-BP data points cross-referenced to timestamps, where the period of time between timestamps is greater than a first period of time, and a sample set is collected for a plurality of physical and mental conditions for the first individual. For example, PWV-to-BP data points may be collected at different times of day, when the first individual has just awakened, or when the first individual is under stress. The physical and mental condition factors can be provided to the screening application 120, for entry into the personal PWV-BP transform file 118, via UI 128. The conditions under which PWV-to-BP data points are collected are of interest due to the cost of the collection and the impact on the ultimate transform. Calibration prompting addresses this problem.

When the personal PWV-BP transform file 118 is determined by the screening application 120 to be valid, it can be used to correlate a current PWV measurement for the first individual 116, to a BP estimate for the first individual. However, the screening application 120 may determine that the personal PWV-BP transform file 118 is invalid if the current PWV measurement fails to meet a statistical limit derived from the current PWV measurement and the plurality of simultaneous PWV measurements and BP measurements for the first individual. That is, the personal PWV-BP transform file is determined to be invalid when the personal PWV-BP transform fails to meet a predetermined statistical limit derived from the plurality of simultaneous PWV measurements and BP measurements for the first individual.

The target measurement use case for the device described above is the home. It is assumed that the collection of calibration points is a burden to the patient (first individual) and those points may sometimes be collected in modes that compromise the integrity of the measurement. Some patients (some of the time) would like to improve the accuracy of their blood pressure estimates and would be willing to accept the burden of occasional calibration to that end. The patient will certainly not accept a rigorous schedule of calibrating with a large number of measurements, however, they may be willing collect a large number of calibration points reflecting great diversity of physical conditions over a long period of time (weeks or months) in return for a stable and accurate blood pressure measurement. This use case dictates the use of robust statistical methods to mitigate measurement error and paucity of data. It also suggests a process which dynamically optimizes the measurement mode to the current measurement and the available calibration data.

The longstanding view of researchers in the area of pulse wave velocity and blood pressure is that the transform from PWV to BP is modeled by the Moens-Koertig equation with elaborations[1, page 66] as:

$${c}_{0} = \sqrt[2]{\frac{{hE}_{0} {e}^{ζ P}}{2 R ρ}} = \sqrt{{se}^{ζ P}} \qquad ( 1 )$$

### For

c₀—pulse wave velocity
h—wall thickness
E₀-13 zero pressure modulus
ζ—vessel constant
P—arterial pressure
R—vessel radius
ρ—blood density

Note that many of these parameters are unobservable even with sophisticated methods, like ultrasound. As such, the parameters may be aggregated as shown in (1) above and a calibration process used to derive the aggregated parameters. Some diversity exists in the literature regarding the specifics of the transform curve. Some authors have suggested nonlinear transforms like the above, while others have suggested linear [2]. The method described here does not assume a specific transform but provides a framework in which linear or non-linear transforms may be applied.

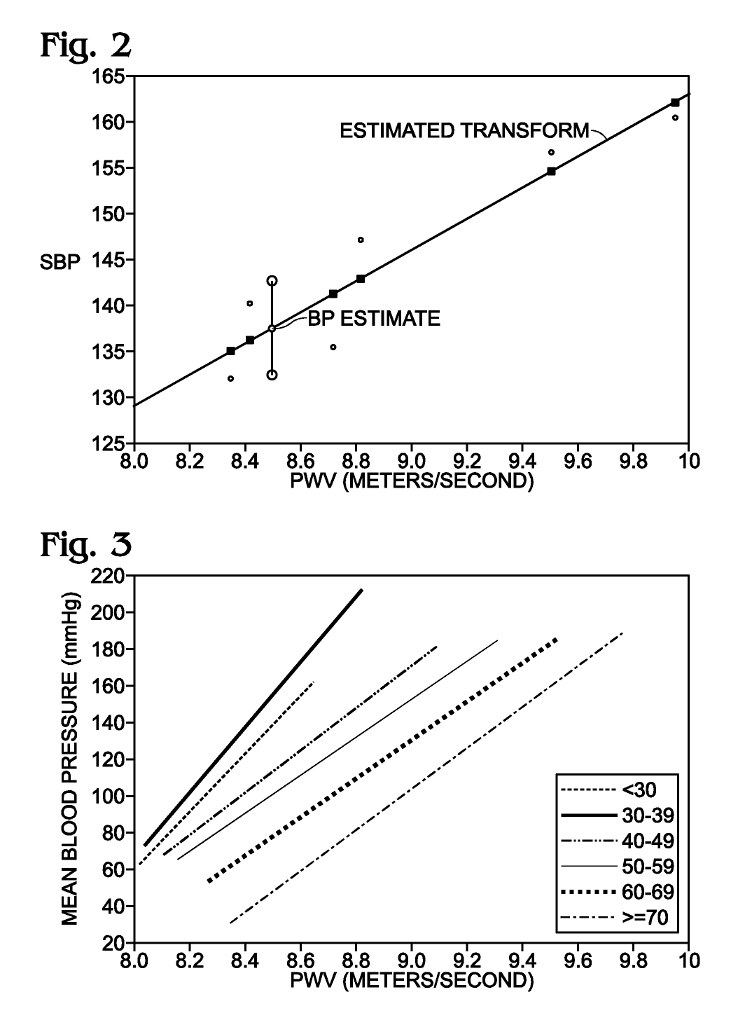

FIG. 2 is a graph of systolic blood pressure (SBP) vs. PWV measurements depicting a calibration set (dots), estimated PWV-to-BP transform, BP estimates (x), and BP estimate with a confidence interval. Characterization of the transform from PWV to BP can be performed by fitting a transform curve to pairs of PWV and BP samples collected from a patient, as illustrated. This fit effectively calibrates the transform curve to a specific patient and, as such, the (PWV, BP) pairs might be called calibration points.

FIG. 3 is a graph depicting a family of transform curves derived from age-decade populations in a large study (n=11,092) [9]. Multipoint calibration can improve BP estimation significantly [2]; in many use cases (home use, for example), while calibration over time is feasible, the points may be derived sporadically and the quality of the calibration measurements may sometimes be poor. Bather than rule out multipoint calibration as generally infeasible, the method described herein takes the view that calibration is a long-term incremental process, spanning days and weeks, and proceeding from low-accuracy population transforms to a more accurate personalized transform. Statistical methods are used to reduce the impact of measurement error and ensure a high-quality transform.

Calibration of the PWV-BP transform overcomes the difficulty that many parameters in the underlying physical model are unobservable. The calibration process “solves” for these parameters without the need of direct observation of their separate values. In addition, it has been found that the actual PWV-BP transform (and parameters) varies over time [3, 4, and 6]. By calibrating over long intervals (e.g. 8 weeks, for example rather than 3 minutes), the estimated transform tends to a form reflecting the expectations of the underlying parameters. As such, long-term calibration may yield a measurement that better reflects the mean state of the patient than one based on short-term calibrations. Of course, the time-varying nature of the actual transform is of concern as variations introduce uncertainty in the measurement. This uncertainty is mitigated by screening the transform and falling back to the population transform in cases whore the personalized transform is unsuitable.

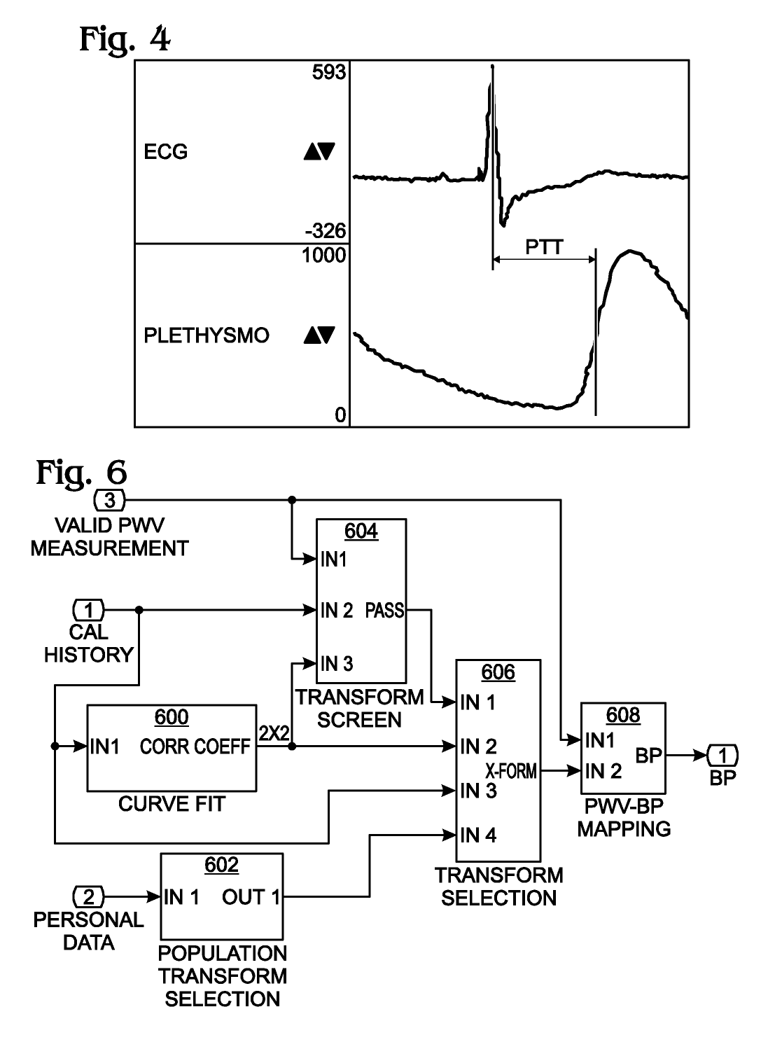

FIG. 4 illustrates waveforms of an exemplary PWV measurement that may be acquired on every arterial pulse [9]. The R-wave of the ECG signal is detected and the “foot” [7,8] of the corresponding Pulse Plethysmography (PPG) signal is detected. The time interval between these detections is measured to estimate the raw, uncorrected arterial Pulse Transit Time (PTT).

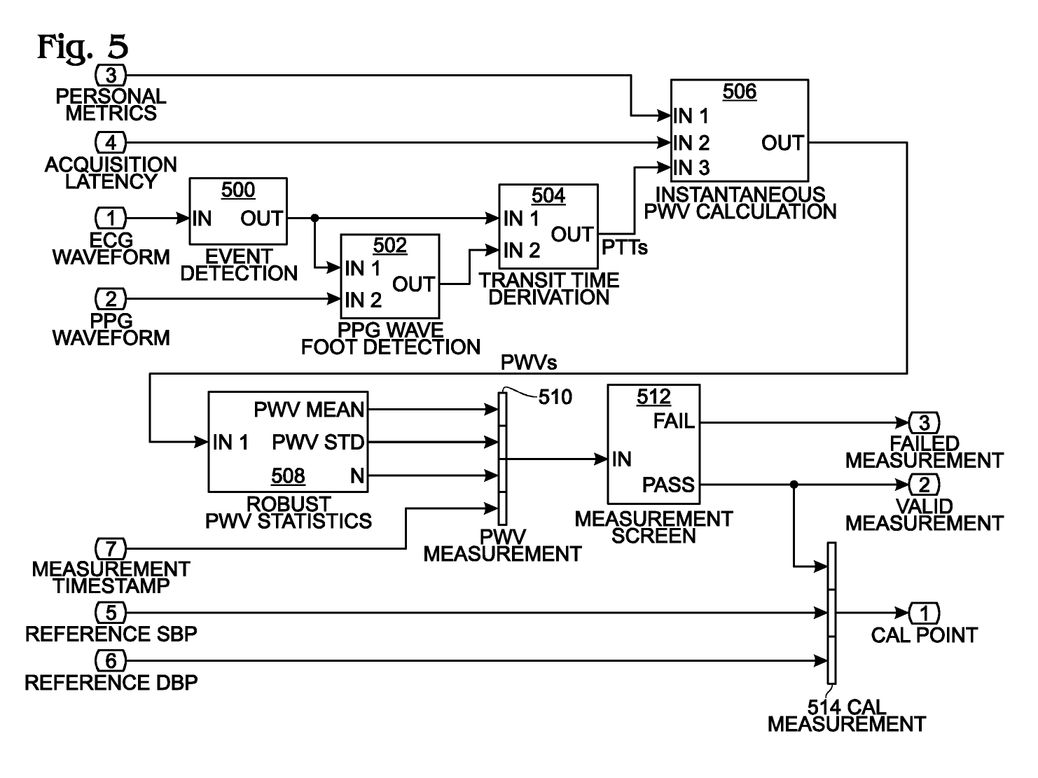

FIG. 5 is a block diagram depicting an exemplary realization of a PWB-BP calibration measurement. It is assumed the PWV measurement is limited by either a fixed time interval (perhaps 30 seconds) or a fixed number of patient pulses (e.g., 30 pulses). The PTT is estimated as previously described (Blocks 500, 502, and 504). Personal metrics (like the distance from center sternum to fingertip, where the PPG signal is collected) and acquisition latency terms dike filter latencies and coronary pro-ejection period (PEP)) are used to correct the raw pulse transit times and transform them into accurate velocity units (Block 506). Acquisition latency is the time between occurrence of a sample and that sample being recorded. If the latency is different for ECG and PPG signals, it may cause an error in the PWV measurement. The PWV sample mean and standard deviation and count are derived from the PWV sequence (Block 508). Robust statistical methods (median filter and uncertainty weighting) may be employed to mitigate the effect of noise on the PWV mean. These PWV statistics are collected with a measurement timestamp to form a PWV measurement (Block 510). A measurement screen (i.e., the screening application) is used to classify the measurement as valid or invalid based on significance (N), uncertainty (STD), and feasibility limits (Block 512). If the PWV measurement is valid, it may be supplemented with reference SBP and diastolic blood pressure (DBP) measurements to yield a calibration measurement (Block 514). Those calibration measurements are then collected into a calibration history or database.

### Transform Derivation

The home use case requires operation with no calibration points, as the patient can track relative trends with only a population transform and may not be concerned about absolute accuracy. However, calibration certainly improves the accuracy of the PWV-BP transform, since a specific patient rarely reflects the population norms. The discussion in this section considers transform derivation from a global perspective and then focuses on a specific mechanism for transform screening and selection. The section concludes with a summary of the transform state over time.

FIG. 6 illustrates the derivation and application of the PWV-BP transform. A fit algorithm (e.g., Uncertainty-Weighted Least Squares) is applied to the calibration history for the patient, resulting in model coefficients (e.g., linear regression coefficients) which define a personalized transform curve (Block 600). The calibration set may be empty, in which case, the result is undefined and will be unused. A population transform baaed on the patient's personal data (e.g., age and gender) is also selected (Block 602). These two transforms, population and personal, form the basis for the final transform. The personal transform is subjected to a statistical screen to determine its viability in the context of the calibration set and the current PWV measurement (Block 604). The two transforms, the result of the screen, and the calibration set are then used to derive the final transform (Block 606) which is then applied to the current PWV measurement to yield a BP-PWV result (Block 608).

Given that patient physiology changes with age and the passage of time in general, a mechanism for eliminating the oldest points from the calibration set is used, subject to a minimum calibration set size. The minimum set size might be the size of the calibration set on first passing the transform screen (as described below). This rule allows the transform to track changes in patient physiology, but retains personalization even if calibrations cease.

An important detail of FIG. 6 is the annotation on the output of Block 600. Two transforms are produced, one for the diastolic and one for the systolic measurement. Both are screened independently (Block 604) but the final transform selection considers the joint screen (Block 606). The next subsection describes the transform screen in detail, followed by a description of the transform selection.

### Transform Screening

Each personalized transform is screened against the calibration set and the current measurement to determine its inferential validity. The screen consists of two hypothesis tests [10, Chapter 8], which are called Test 1 and Test 2. Test 1 establishes the validity of the transform model itself in the context of the calibration set. Test 2 establishes the validity of the transform relative to the current measurement and calibration set. Both are structured as tests of a Null (or basis) Hypothesis. The meaning associated with each Null Hypothesis is that the population transform is the appropriate transform. The logical complement of the Null Hypothesis is the Alternative Hypothesis, associated with the meaning that the personalized transform is the appropriate transform.

Test 1 uses a well known statistical procedure called a Model Utility Test [10, Chapter 12]. The Test 1 Null Hypothesis is that the transform slope β₁ has value of zero relative to the estimated standard error of the slope. A high level of confidence is required to reject that hypothesis (e.g., 99%). In practice, a calibration set with few points or points with little diversity relative to the variability of the estimates will fail to reject the hypothesis. Greater point count and diversity favor rejection of the hypothesis. As such, the inferential validity of the transform is established.

In more detail. Test 1 relies on the standardized variable:

$$t = \frac{{\overline{β}}_{1} - {β}_{1}}{{s}_{{\hat{β}}_{1}}} \qquad ( 1 )$$

where β₁ is the trite regression slope, \hat{β}₁ is the estimated slope, and s_{\hat{β}}_{₁} is the estimated standard deviation of the slope. The quantity \hat{β}₁-β₁ is an estimate residual, the difference between an estimated value and its true value. The hypothesis assumes a slope of zero, so (1) reduces to:

$$t = \frac{{\hat{β}}_{1}}{{s}_{{\hat{β}}_{2}}} \qquad ( 2 )$$

The estimated standard deviation of the slope (S_{\hat{β}}_{₁}) is calculated as:

$${S}_{{\hat{β}}_{1}} = \frac{s}{\sqrt{{S}_{xx}}} = \frac{\sqrt{\frac{SSE}{n - 2}}}{\sqrt{\sum {( {x}_{i} - \overline{x} )}^{2}}} \qquad ( 3 )$$

where SSE is defined as:

SSE=Σ(yᵢ−ŷᵢ)² (4)

The numerator of (3) is the sample standard deviation of the estimate. The term (n−2) represents the degrees of freedom associated with the sum of squared errors (SSE) of the transform applied to the calibration set. The SSE is simply a squaring and summing of the residuals of the transform over the calibration sot.

The Test 1 hypothesis states that:

−t_{α/2,n-2}<t<t_{α/2,n-2} (4)

where t_{α/2,n-2} is the value of the Student's T distribution CDF at α/2 for n−2 degrees of freedom. The quantity t has a Student T distribution with n−2 degrees of freedom. Equation 4 establishes a confidence interval about the ratio t. The statistical interpretation of (4) is that ever many trials, the true (unobservable) value of t lies within the stated interval with probability 1−α (e.g., 90%). Since the hypothesis is stated with a wide margin, the failure of (4) provides strong evidence that the slope of the transform is non-zero, which implies the utility of the linear model in characterizing the calibration set and implies the inferential validity of the resulting transform.

As an example, consider a calibration set with the following PWV and SBP values:

TABLE 1
A small calibration set

| PWV | SBP |
|---|---|
| 8.42 | 140 |
| 8.71 | 135 |
| 8.81 | 147 |
| 8.35 | 132 |

A 99% confidence-level yields the following relations from (4):

−t_{0.005,2}=−9.9248<0.6996<9.9248=t_{0.005,2} (5)

Hence, Test 1 “fails to reject” it's Null Hypothesis with the meaning that the generated transform lacks inferential validity.

For a larger data set:

TABLE 2
A larger calibration set

| PWV | SBP |
|---|---|
| 8.42 | 140 |
| 8.71 | 135 |
| 8.81 | 147 |
| 8.35 | 132 |
| 9.95 | 161 |
| 9.50 | 157 |

Table 2 yields the following relations from (4) at 99% confidence level:

−t_{0.00544}=−4.6041<5.26≧4.6041=t_{0.005,4} (6)

Because t=5.26 exceeds the upper bound of the confidence interval, the Null hypothesis is rejected. This provides strong evidence that the slope parameter is non-zero and the inferential validity of the transform is thereby established.

The transform derived from the calibration set can be expected to perform better for PWV measurements close to the mean PWV of the calibration set than measurements far away. However, Test 1 does not consider the current measurement. Test 2 performs that function. Teat 2 relies on the following definition:

$$t = \frac{\hat{Y} - ( {β}_{0} + {β}_{1} {x}^{*} )}{{S}_{\hat{Y}}} \qquad ( 7 )$$

This is the residual normalized by the sample standard deviation of the estimate. Ŷ is the estimate (i.e., blood pressure), x* is the current PWV measurement, S_{Ŷ} is the sample standard deviation of the estimate, and βᵢ are the true (and unobservable) regression coefficients. The variable has Student's T distribution with n−2 degrees of freedom.

A confidence interval around the current estimate is:

y±t_{α/2,n-2}·S_{Ŷ} (8)

where

$${S}_{\hat{Y}} = \sqrt[s]{\frac{1}{n} + \frac{{( {x}^{*} - \overline{x} )}^{2}}{{s}_{xx}}} \qquad ( 9 )$$

for variables defined above.

Test 2 sets a confidence interval at some level (e.g., 90%) and compares the resulting offset t_{α/2,n-2}·S_{Ŷ} to a blood pressure threshold. The Null hypothesis is:

t_{α/2,n-2}·S_{Ŷ}>thresh (10)

If (10) does not hold, the Null Hypothesis is rejected and the inferential validity of the transform for the current point is established.

As an example of Test 2, consider the calibration set in Table 2, a PWV measurement of 8.5 meters per second (m/s), and a confidence level of 90%. The blood pressure prediction is 138 millimeter of mercury (mmHg), S_{Ŷ}=2.378 m/s, and t_{0.005,4}=2.132. From (10), for threshold of 10 mmHg, it is given that:

t_{α/2,n-2}·S_{Ŷ}=(2.132×2.378)=5.07<10 (11)

As such, the Null Hypothesis is rejected and the Alternative Hypothesis is established, from which it may be inferred that the personalized transform is valid for the measured PWV. In this example, as the PWV distance from the mean calibration PWV (e.g., x*−x̅) (9) increases the LHS of (10) increases, eventually causing the Null

Hypothesis to hold, from which it may be inferred that the personalized transform is not valid for the current PWV measurement.

If the Alternative Hypothesis is established for both Test 1 and Test 2. the personalized transform passes the screen and may be used to estimate blood pressure.

### Transform Selection

With the completion of Blocks 600, 6102, and 604 of FIG. 6, two transforms are available (population and personal), along with the personal transform screen result and the calibration set. Transform selection might simply select the personal transform if it passed the screen and otherwise select the population transform. However, the population transform is a gross approximation. Even if the screen fails, it can be improved by adding a correction term which translates the population transform to pass through the mean calibration point. That correction term is simply the mean calibration blood pressure less the mean estimate under the population curve. In essence, this is a simple “calibration” of the population curve to the patient:

c=y̅−(\hat{β}_{α}+\hat{β}₁x) (12)

where y̅ is the mean calibration blood pressure, x̅ in the mean calibration PWV, and \hat{β}ᵢ represent the population transform.

The transform selection can now be stated simply: If the size of the calibration set is at least 1, adjust the population transform by adding the correction term of (12). If the personal transform fails the screen, then select the adjusted population transform. Otherwise, select the personal transform.

### Progression or Transform State

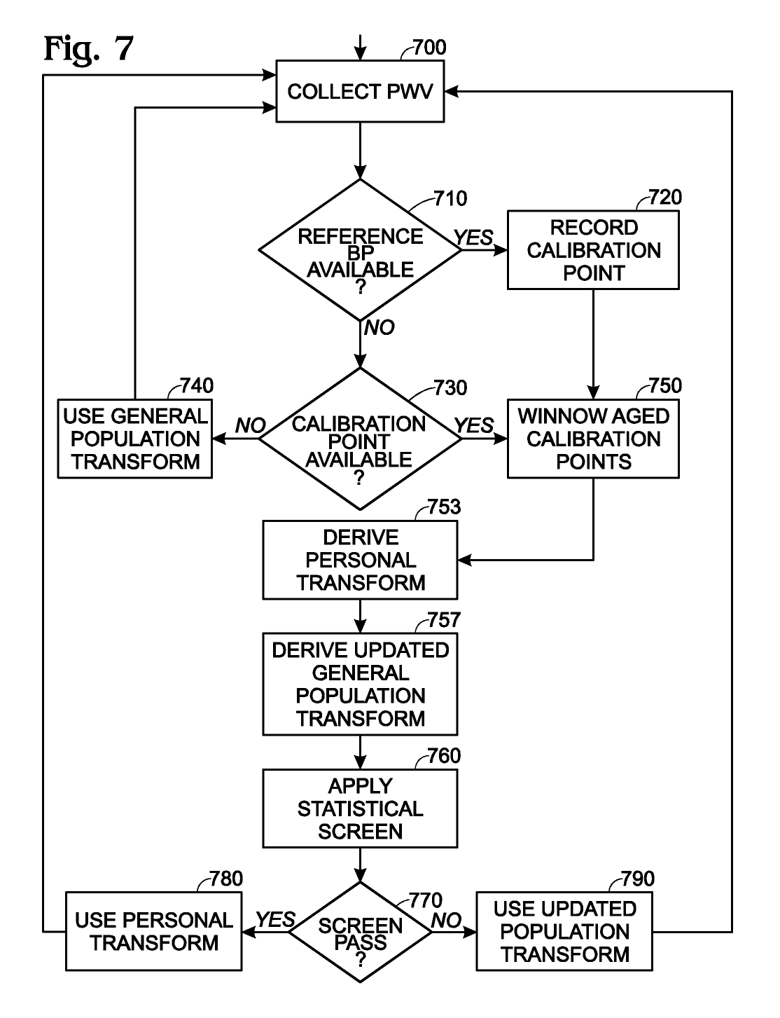

FIG. 7 is a flowchart, illustrating a method for deriving a PWV-BP transform. A PWV measurement is collected from the device (Step 700). Initially, no calibration points have been collected and it is assumed that no reference BP measurement is available. In this case, the initial transform is chosen based on population indices alone (Step 740). This transform may be used indefinitely to transform PWV measurements to BP. However, to improve measurement performance, calibration points may be collected. A PWV measurement is collected and an associated reference BP measurement is also collected. The PWV and reference measurements are recorded as a calibration point (Step 720). Once a calibration point has been collected, on any new measurement, the calibration set is updated to remove very old calibration points (Step 750). The personal transform (Step 753) and updated general population transforms (Step 757) are derived from the calibration set. Next, statistical screening is applied (Step 760). If it passes (Step 770), then the calibration points are fit to a transform curve and that curve serves as the personalized transform and is applied to yield a blood pressure. If the screen fails, a correction term is derived from the calibration points and applied to the population transform as an approximate personalization. That updated general population transform is used to yield a blood pressure estimate (Step 790).

Step 740 provides a general population PWV-BP transform comprising an average population correlation of PWV measurements to BP measurements, where a selected average population shares common characteristics with a selected first individual. Step 700 takes a current PWV measurement and Step 760 determines the validity of a personal PWV-BP transform. The personal PWV-BP transform is based upon PWV and BP measurements for the first individual. When the personal PWV-BP transform is determined to be invalid. Step 790 uses the general population PWV-BP to correlate the current PWV measurement for the first individual, to an estimated BP value for the first individual.

In one aspect, Step 720 simultaneously takes at least a first PWV measurement and a first BP measurement for the first individual. Step 757 modifies the general population PWV-BP transform using the at least the first PWV measurement and the first BP measurement. Step 757 also saves the modified general population PWV-BP transform as an updated general population PWV-BP transform. Step 720 takes a plurality of simultaneous PWV measurements and BP measurements for the first individual. Step 780 establishes the personal PWV-BP transform in response to the plurality of PWV and BP measurements. In one aspect, Step 780 establishes the personal PWV-BP transform by collecting a PWV measurement as a statistical mean over many arterial pulses within the time interval of the PWV measurement.

In one aspect, when the personal PWV-BP transform is determined to be valid in Steps 760 and 770, Step 780 uses the personal PWV-BP transform to correlate a current PWV measurement for the first individual, to the BP estimate for the first individual. In another aspect, Steps 760 and 770 determines the personal PWV-BP transform to be invalid when the current PWV measurement fails to meet a statistical limit derived from the current PWV measurement for the first individual and the plurality of simultaneous PWV measurements and BP measurements for the first individual taken in Step 720. For example, Steps 760 and 770 may determine personal PWV-BP transform invalidity when the plurality of simultaneous PWV measurements and BP measurements for the first individual fails to meet a statistical limit.

In another aspect, Step 720 collects a plurality of PWV-to-BP data points, cross-referenced to timestamps. Then, Step 753 establishes the personal PWV-BP transform in consideration of the PWV-to-BP data point timestamps, and a PWV-to-BP data point count. In one aspect, Step 720 also collects a sample set with PWV-to-BP data points, cross-referenced to timestamps, where the period of time between timestamps is greater than a first period of time. In another aspect, in Step 720, PWV-to-BP data point sample sets may be collected for a plurality of physical and mental conditions for the first individual.

A final example, tied to the examples of the previous sections, may make the algorithm and its application more concrete. Luci Smith has purchased a PWV-BP monitor for the purpose of taking quick measurements on her commute to and from work on the train. On her ride home, she removes the new device from a coat pocket, enters her personal data—age, gender, height, weight—and takes a measurement. The device uses a transform baaed on her personal data to produce a blood pressure estimate. It also advises Luci that measurement accuracy might be improved through personal calibration. On arriving home, she takes another PWV-BP measurement along with a cuff BP measurement, which she enters into the device. The device uses the calibration measurement to personalize her population transform (equation 12) and displays the calibrated measurement. Luci continues to calibrate the device in her spare time at home. Two days later she sees her accumulated calibration set, shown in Table 1. Her calibration statistics are not yet convincing and her measurements on the train are still transformed using the population transform with simple personalization. Two additional calibrations yield the calibration set shown in Table 2. Next morning on the train, she measures her blood pressure with her PWV-BP device. Her PWV is 6.969 m/s. Since her calibration data now have a convincing statistical basis and the PWV value is also in a range which is appropriate for inference, the device selects her personalized PWV-BP transform and produces an SBP estimate of 136 mmHg. At work, she is stressed and fatigued. On her way home, she measures her blood pressure. Her PWV is 8.5 m/s. Since there is no statistical basis for transforming this measurement using her accumulated calibration set, the device uses her corrected population transform to produce her SBP estimate. An additional calibration that evening improves the range of her calibration set and the subsequent measurement is transformed using a new, refined personalized transform.

The novelty of this approach derives from the application of well-founded statistical methods to the incremental evolution of the PWV-BP transform from calibration sets derived over long time intervals. This approach is well-adapted to the home use case. The measurement is not limited to population norms but benefits from personalization where the sample statistics are supportive. No requirement is asserted to calibrate often, or at all, or on any schedule. Rather, calibration is accepted as it becomes available and is used to personalize the PWV-BP transform to the greatest extent supported by the sample statistics.

A system and method have been provided for deriving a PWV-BP transform. Examples of particular statistical processes have been presented to illustrate the invention. However, the invention is not limited to merely these examples. Other variations and embodiments of the invention will occur to those skilled in the art.

We claim:

1. A method for deriving a pulse wave velocity-blood pressure (PWV-BP) transform, the method comprising:
providing a general population PWV-BP transform comprising an average population correlation of PWV measurements to BP measurements, where a selected average population shares common characteristics with a selected first individual;
determining the validity of a personal PWV-BP transform, where the personal PWV-BP transform is based upon PWV and BP measurements for the first individual; and,
when the personal PWV-BP transform is determined to be invalid, using the general population PWV-BP to correlate a current PWV measurement for the first individual, to an estimated BP value for the first individual.

2. The method of claim 1 further comprising:
simultaneously taking at least a first PWV measurement and a first BP measurement for the first individual;
modifying the general population PWV-BP transform using the at least the first PWV measurement and the first BP measurement; and,
saving the modified general population PWV-BP transform as an updated general population PWV-BP transform.

3. The method of claim 2 further comprising:
taking a plurality of simultaneous PWV measurements and BP measurements for the first individual; and,
establishing the personal PWV-BP transform in response to the plurality of PWV and BP measurements.

4. The method of claim 3 further comprising:
when the personal PWV-BP transform is determined to be valid, using the personal PWV-BP transform to correlate the current PWV measurement for the first individual, to the BP estimate for the first individual.

5. The method of claim 4 wherein determining the validity of the personal PWV-BP transform includes determining invalidity when the current PWV measurement fails to meet a statistical limit derived from the current PWV measurement for the first individual and the plurality of simultaneous PWV measurements and BP measurements for the first individual.

6. The method of claim 5 wherein determining the validity of the personal PWV-BP transform includes determining invalidity when the plurality of simultaneous PWV measurements and BP measurements for the first individual fail to meet a statistical limit.

7. The method of claim 6 wherein taking the plurality of simultaneous PWV measurements and BP measurements for the first individual includes collecting a plurality of PWV-to-BP data points, cross-referenced to timestamps; and,
wherein establishing the personal PWV-BP transform includes establishing the personal PWV-BP transform in consideration of the PWV-to-BP data point timestamps, and a PWV-to-BP data point count.

8. The method of claim 3 wherein establishing the personal PWV-BP transform includes collecting a PWV measurement as a statistical mean over many arterial pulses within the time interval of the PWV measurement.

9. The method of claim 8 wherein taking the plurality of simultaneous PWV measurements and BP measurements for the first individual includes collecting a sample set with PWV-to-BP data points, cross-referenced to timestamps, where the period of time between timestamps is greater than a first period of time.

10. The method of claim 9 wherein collecting the plurality of PWV-to-BP data points includes collecting PWV-to-BP data points for a plurality of physical and mental conditions for the first individual.

11. A device for correlating pulse wave velocity (PWV) and blood pressure (BP) measurements, the device comprising:
a PWV measurement interface comprising an electrocardiogram (ECG) sensor and a photoplethysmography (PPG) sensor for measuring ECG and PPG signals;
a processor:
a non-transitory memory including:
a general population PWV-BP transform file with an average population correlation of PWV measurements to BP measurements, where a selected average population shares common characteristics with a selected first individual;
a personal PWV-BP transform file, where the personal PWV-BP transform file is based upon PWV and BP measurements for a first individual; and,
a screening application enabled as a sequence of processor instructions for determining the validity of the personal PWV-BP transform file, the screening application selecting the general population PWV-BP transform file when the personal PWV-BP transform file is determined to be invalid, and using the general population PWV-BP file to correlate a current PWV measurement for the first individual, to an estimated BP value for the first individual.

12. The device of claim 11 further comprising:
a BP port for accepting BP measurements;
wherein the screening application accepts data for at least a first PWV measurement and a first BP measurement, simultaneously taken for the first individual, and creates an updated general population PWV-BP transform file; and,
the non-transitory memory further comprising:
the updated general population PWV-BP transform file including the general population PWV-BP transform file as modified by at least the first PWV measurement and the first BP measurement.

13. The device of claim 12 wherein the screening application accepts data for a plurality of simultaneous PWV measurements and BP measurements taken for the first individual, and creates the personal PWV-BP transform file in response to the plurality of PWV and BP measurements.

14. The device of claim 13 wherein the screening application uses the personal PWV-BP transform file to correlate the current PWV measurement for the first individual, to a BP estimate for the first individual, when the personal PWV-BP transform file is determined to be valid.

15. The device of claim 14 wherein the screening application determines the personal PWV-BP transform file to be invalid when the current PWV measurement fails to meet a statistical limit derived from the current PWV measurement and the plurality of simultaneous PWV measurements and BP measurements for the first individual.

16. The device of claim 15 wherein the screening application determines the personal PWV-BP transform file to be invalid when the personal PWV-BP transform fails to meet a predetermined statistical limit derived from the plurality of simultaneous PWV measurements and BP measurements for the first individual.

17. The device of claim 13 wherein the personal PWV-BP transform fib includes a plurality of PWV-to-BP data points, cross-referenced to timestamps; and,
wherein the screening application creates the personal PWV-BP transform file in response to the PWV-to-BP data point timestamps and a PWV-to-BP data point count.

18. The device of claim 13 wherein the screening application creates the personal PWV-BP transform file by collecting a PWV measurement as a statistical mean over many arterial pulses within the time interval of the PWV measurement.

19. The device of claim 13 wherein the personal PWV-BP transform file includes a PWV-to-BP sample set, with PWV-to-BP data points cross-referenced to timestamps, where the period of time between timestamps is greater than a first period of time: and,
wherein a sample set is collected for a plurality of physical and mental conditions for the first individual.
